(19) United States
(12) Patent Application Publication
(10) Pub. No.: US 2024/0153637 A1
(43) Pub. Date: May 9, 2024

(54) MEDICAL SUPPORT DEVICE, OPERATION METHOD OF MEDICAL SUPPORT DEVICE, OPERATION PROGRAM OF MEDICAL SUPPORT DEVICE, LEARNING DEVICE, AND LEARNING METHOD

(71) Applicant: FUJIFILM Corporation, Tokyo (JP)

(72) Inventor: Caihua WANG, Kanagawa (JP)

(73) Assignee: FUJIFILM Corporation, Tokyo (JP)

(21) Appl. No.: 18/544,307

(22) Filed: Dec. 18, 2023

Related U.S. Application Data
(63) Continuation of application No. PCT/JP2022/025624, filed on Jun. 27, 2022.

(30) Foreign Application Priority Data
Jun. 28, 2021 (JP) 2021-106861

(51) Int. Cl. G16H 50/20; G16H 10/60; G16H 50/50

(52) U.S. Cl. CPC G16H 50/20; G16H 10/60; G16H 50/50

(57) ABSTRACT

A medical support device includes: a processor; and a memory connected to or built into the processor, in which the processor is configured to: acquire target input data which is input data related to a disease of a subject whose progression of the disease is to be predicted, and a prediction interval which is an interval from a reference point in time to a future point in time at which prediction is performed; and input the target input data and the prediction interval to a machine learning model trained using supervised training data including accumulated input data related to a disease at two or more points in time and a time interval of the input data, and cause the machine learning model to output a prediction result regarding the disease of the subject at the future point in time.

### CROSS-REFERENCE TO RELATED APPLICATIONS

This application is a continuation application of International Application No. PCT/JP2022/025624 filed on Jun. 27, 2022, the disclosure of which is incorporated herein by reference in its entirety. Further, this application claims priority from Japanese Patent Application No. 2021-106861 filed on Jun. 28, 2021, the disclosure of which is incorporated herein by reference in its entirety.

### BACKGROUND

### 1. Technical Field

A technology of the present disclosure relates to a medical support device, an operation method of a medical support device, an operation program of a medical support device, a learning device, and a learning method.

### 2. Description of the Related Art

With the advent of a full-fledged aging society, it is becoming increasingly important to accurately predict the progression of diseases such as dementia represented by Alzheimer's dementia and to establish optimal treatment policies according to the prediction. However, there are many parts of dementia that have not yet been elucidated pathologically, and there are various factors involved in the deterioration of symptoms. Therefore, it has been difficult to accurately predict the progression of dementia with simple prediction models in the related art such as mathematical models. Therefore, in recent years, research has been actively conducted in which a machine learning model that outputs prediction results for unknown input data by learning a large amount of supervised training data predicts the progression of dementia.

For example, “M. Nguyen, T. He and L. An et al.: Predicting Alzheimer's disease progression using deep recurrent neural networks, NeuroImage, Nov. 2020” (hereinafter referred to as Document 1) discloses a technology for predicting the progression of dementia using a recurrent neural network (hereinafter abbreviated as RNN) as a machine learning model. In Document 1, test data related to dementia at three or more points in time (for example, test data three months ago, two months ago, and one month ago) is given to the RNN as a set of supervised training data for learning.

### SUMMARY

The number of donors of test data related to dementia is less than 3,000 even in Alzheimer's disease Neuroimaging Initiative (ADNI), which is the most popular database. That is, in the method of Document 1, the amount of supervised training data is significantly insufficient. Therefore, in the method of Document 1, there is a concern that overlearning may occur and the accuracy of predicting the progression of dementia may be significantly reduced.

An embodiment according to the technology of the present disclosure provides a medical support device, an operation method of a medical support device, an operation program of a medical support device, a learning device, and a learning method that can suppress a decrease in accuracy of predicting the progression of a disease.

According to an aspect of the present disclosure, there is provided a medical support device comprising: a processor; and a memory connected to or built into the processor, in which the processor is configured to: acquire target input data which is input data related to a disease of a subject whose progression of the disease is to be predicted, and a prediction interval which is an interval from a reference point in time to a future point in time at which prediction is performed; and input the target input data and the prediction interval to a machine learning model trained using supervised training data including accumulated input data related to a disease at two or more points in time and a time interval of the input data, and cause the machine learning model to output a prediction result regarding the disease of the subject at the future point in time.

It is preferable that the input data includes at least one of test data indicating a result of a test related to a disease or diagnostic data indicating a result of a diagnosis related to the disease.

It is preferable that the target input data includes data at a current point in time of the subject, and the reference point in time includes the current point in time.

It is preferable that the target input data includes data at a past point in time of the subject, and the reference point in time includes the past point in time.

It is preferable that the processor is configured to, in a case where a plurality of pieces of the target input data and a plurality of the prediction intervals corresponding to a plurality of the reference points in time are acquired, cause the machine learning model to output a plurality of the prediction results for each of the plurality of pieces of target input data and the plurality of prediction intervals, and derive an integrated prediction result in which the plurality of prediction results are integrated.

It is preferable that the processor is configured to derive an arithmetic mean of the plurality of prediction results as the integrated prediction result.

It is preferable that the processor is configured to derive a weighted average of the plurality of prediction results as the integrated prediction result.

It is preferable that the processor is configured to change weights given to the plurality of prediction results in a case where the weighted average is calculated, according to the prediction interval.

It is preferable that the processor is configured to set the weights given to the plurality of prediction results in the case where the weighted average is calculated, using a function having the prediction interval as a variable.

It is preferable that the disease is dementia.

According to another aspect of the present disclosure, there is provided an operation method of a medical support device, the method comprising: acquiring target input data which is input data related to a disease of a subject whose progression of the disease is to be predicted, and a prediction interval which is an interval from a reference point in time to a future point in time at which prediction is performed; and inputting the target input data and the prediction interval to a machine learning model trained using supervised training data including accumulated input data related to a disease at two or more points in time and a time interval of the input data, and causing the machine learning model to output a prediction result regarding the disease of the subject at the future point in time.

According to another aspect of the present disclosure, there is provided an operation program of a medical support device causing a computer to execute a process comprising: acquiring target input data which is input data related to a disease of a subject whose progression of the disease is to be predicted, and a prediction interval which is an interval from a reference point in time to a future point in time at which prediction is performed; and inputting the target input data and the prediction interval to a machine learning model trained using supervised training data including accumulated input data related to a disease at two or more points in time and a time interval of the input data, and causing the machine learning model to output a prediction result regarding the disease of the subject at the future point in time.

According to another aspect of the present disclosure, there is provided a learning device that performs learning, the learning device being configured to, using at least accumulated input data related to a disease at two or more points in time and a time interval of the input data, as supervised training data, and using target input data which is input data related to a disease of a subject whose progression of the disease is to be predicted, and a prediction interval which is an interval from a reference point in time to a future point in time at which prediction is performed, as inputs, learn to obtain a prediction result regarding the disease of the subject at the future point in time, as an output.

According to another aspect of the present disclosure, there is provided a learning method comprising: learning, using at least accumulated input data related to a disease at two or more points in time and a time interval of the input data, as supervised training data, and using target input data which is input data related to a disease of a subject whose progression of the disease is to be predicted, and a prediction interval which is an interval from a reference point in time to a future point in time at which prediction is performed, as inputs, to obtain a prediction result regarding the disease of the subject at the future point in time, as an output.

According to the technology of the present disclosure, it is possible to provide a medical support device, an operation method of a medical support device, an operation program of a medical support device, a learning device, and a learning method that can suppress a decrease in accuracy of predicting the progression of a disease.

### BRIEF DESCRIPTION OF THE DRAWINGS

Exemplary embodiments according to the technique of the present disclosure will be described in detail based on the following figures, wherein:

FIG. 1 is a diagram showing a dementia progression prediction server and a user terminal;

FIG. 2 is a diagram showing target input data;

FIG. 3 is a diagram showing a prediction interval;

FIG. 4 is a diagram showing a progression prediction result;

FIG. 5 is a block diagram showing a computer constituting the dementia progression prediction server;

FIG. 6 is a block diagram showing a processing unit of a CPU of the dementia progression prediction server;

FIG. 7 is a block diagram showing a detailed configuration of a dementia progression prediction model;

FIG. 8 is a diagram showing an outline of processing in a learning phase of the dementia progression prediction model;

FIG. 9 is a diagram for describing the formation of supervised training data of the dementia progression prediction model;

FIG. 10 is a diagram for describing another example of the formation of supervised training data of the dementia progression prediction model;

FIG. 11 is a diagram showing an outline of processing in an operation phase of the dementia progression prediction model;

FIG. 12 is a diagram showing a dementia progression prediction screen;

FIG. 13 is a diagram showing a dementia progression prediction screen on which a message indicating a progression prediction result is displayed;

FIG. 14 is a flowchart showing a processing procedure of the dementia progression prediction server;

FIG. 15 is a diagram showing an aspect in which a plurality of prediction intervals with a current point in time as a reference point in time are input to a dementia progression prediction model, and a plurality of progression prediction results are output from the dementia progression prediction model;

FIG. 16 is a diagram showing a second embodiment in which target input data at a past point in time and a prediction interval with the past point in time as a reference point in time are input to a dementia progression prediction model, and a progression prediction result is output from the dementia progression prediction model;

FIG. 17 is a diagram showing a third embodiment in which a plurality of pieces of target input data at a past point in time and a current point in time and a plurality of progression prediction results for each of a plurality of prediction intervals with the past point in time and the current point in time as reference points in time are output from a dementia progression prediction model and an integrated progression prediction result in which the plurality of progression prediction results are integrated is derived;

FIG. 18 is a diagram showing another example of a progression prediction result;

FIG. 19 is a diagram showing one aspect of a fourth embodiment in which an arithmetic mean of a plurality of progression prediction results is used as an integrated progression prediction result;

FIG. 20 is a diagram showing one aspect of the fourth embodiment in which a weighted average of a plurality of progression prediction results is used as an integrated progression prediction result;

FIG. 21 is a diagram showing another example of a score prediction result;

FIG. 22 is a diagram showing still another example of a score prediction result;

FIG. 23 is a graph showing a Gaussian function for setting weights given to a plurality of score prediction results; and

FIG. 24 is a diagram showing a fifth embodiment in which a weighted average of a plurality of score prediction results is used as an integrated score prediction result.

### DETAILED DESCRIPTION

### First Embodiment

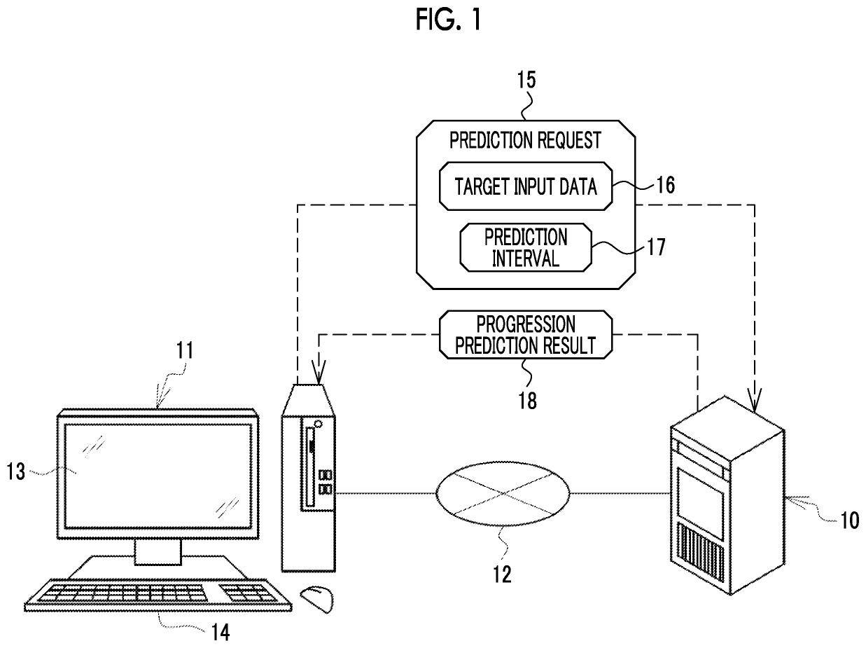

As shown in FIG. 1 as an example, a dementia progression prediction server 10 is connected to a user terminal 11 via a network 12. The dementia progression prediction server 10 is an example of a “medical support device” according to the technology of the present disclosure. The user terminal 11 is installed in, for example, a medical facility, for example, and is operated by a doctor who diagnoses dementia, particularly Alzheimer's dementia, at the medical facility. Examples of dementia include Lewy body dementia, vascular dementia, and the like, in addition to Alzheimer's dementia. The content of the diagnosis may be Alzheimer's disease other than Alzheimer's dementia. Specifically, examples thereof include a preclinical Alzheimer's disease (PAD) and mild cognitive impairment (MCI) due to Alzheimer's disease. Hereinafter, Alzheimer's disease is sometimes abbreviated as AD. The disease is preferably a brain disease such as dementia as an example. The user terminal 11 includes a display 13 and an input device 14 such as a keyboard and a mouse. The network 12 is, for example, a wide area network (WAN) such as the Internet or a public communication network. Although only one user terminal 11 is connected to the dementia progression prediction server 10 in FIG. 1, in practice, a plurality of user terminals 11 of a plurality of medical facilities are connected to the dementia progression prediction server 10.

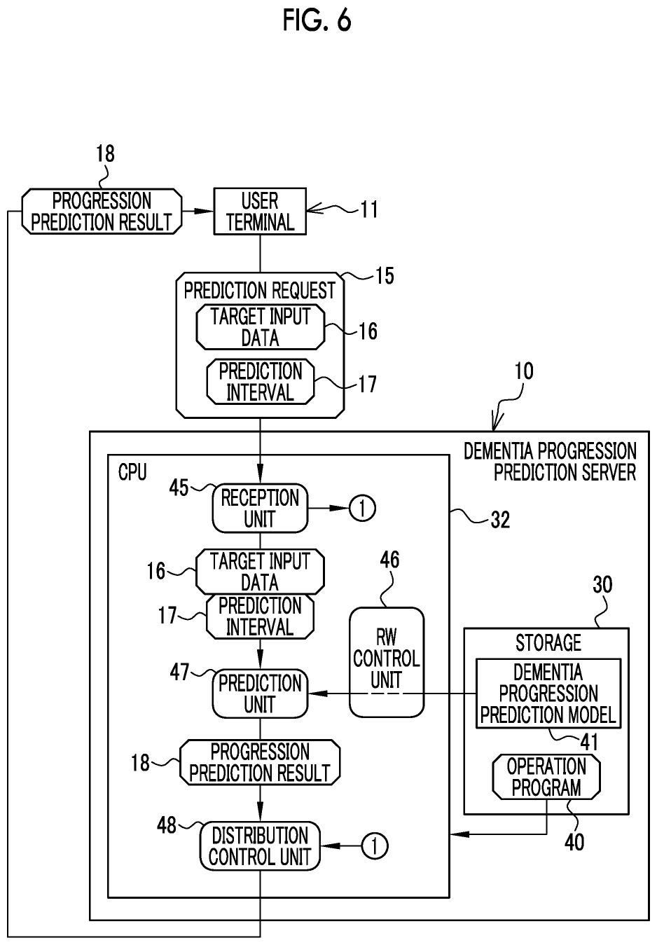

The user terminal 11 transmits a prediction request 15 to the dementia progression prediction server 10. The prediction request 15 is a request for causing the dementia progression prediction server 10 to predict the progression of dementia using a dementia progression prediction model 41 (refer to FIG. 6). The prediction request 15 includes target input data 16 and a prediction interval 17. The target input data 16 is data related to dementia of a subject whose progression of dementia is to be predicted, and is preferably data related to diagnostic criteria for dementia.

Diagnostic criteria for dementia include the diagnostic criteria described in “Dementia disease medical care guideline 2017” supervised by the Japanese Society of Neurology, “International Statistical Classification of Diseases and Related Health Problems (ICD)-11 (ICD-11)”, the American Psychiatric Association's “Diagnostic and Statistical Manual of Mental Disorders, Fifth Edition (DSM-5)”, and the “National Institute on Aging-Alzheimer's Association workgroup (NIA-AA) criteria”. Such diagnostic criteria can be cited, and the contents thereof are incorporated in the present specification.

Examples of data related to the diagnostic criteria for dementia include the data related to the above-described diagnostic criteria. The target input data 16 includes data related to diagnostic criteria for dementia. Specifically, data related to diagnostic criteria for dementia includes cognitive function test data, morphological image test data, brain function image test data, blood/cerebrospinal fluid test data, genetic test data, and the like. The target input data 16 preferably includes at least the morphological image test data, and more preferably includes at least the morphological image test data and the cognitive function test data.

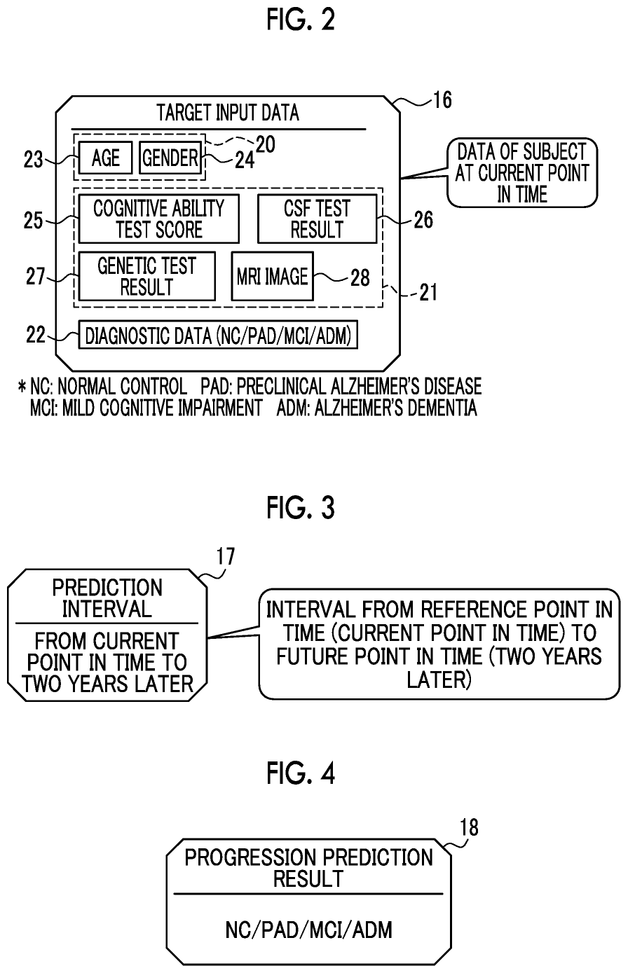

Cognitive function test data includes a clinical dementia rating-sum of boxes (hereinafter abbreviated as CDR-SOB) score, a mini-mental state examination (hereinafter abbreviated as MMSE) score, an Alzheimer's disease assessment scale-cognitive subscale (hereinafter abbreviated as ADAS-Cog) score, and the like. The morphological image test data includes a brain tomographic image obtained by magnetic resonance imaging (MRI) (hereinafter referred to as an MRI image) 28 (refer to FIG. 2), a tomographic image of the brain obtained by computed tomography (CT), and the like.

The brain function image test data include a tomographic image of the brain obtained by a positron emission tomography (PET) (hereinafter referred to as a PET image), a tomographic image of the brain obtained by a single photon emission computed tomography (SPECT), an image (hereinafter referred to as a SPECT image), and the like. The blood/cerebrospinal fluid test data includes an amount of phosphorylated tau protein (p-tau) 181 in cerebrospinal fluid (hereinafter abbreviated as CSF), and the like. The genetic test data includes a test result of a genotype of an ApoE gene, and the like.

The target input data 16 is input by a doctor operating the input device 14. The prediction interval 17 is an interval from the reference point in time to a future point in time at which the progression of dementia is to be predicted, and is also input by the doctor operating the input device 14. Although not shown, the prediction request 15 also includes a terminal ID (identification data) or the like for uniquely identifying the user terminal 11 that is a transmission source of the prediction request 15.

In a case where the prediction request 15 is received, the dementia progression prediction server 10 inputs the target input data 16 and the prediction interval 17 to the dementia progression prediction model 41, and causes the dementia progression prediction model 41 to output a prediction result of progression (hereinafter referred to as a progression prediction result) 18 of the dementia. The dementia progression prediction server 10 distributes the progression prediction result 18 to the user terminal 11 that is a transmission source of the prediction request 15. In a case where the progression prediction result 18 is received, the user terminal 11 displays the progression prediction result 18 on the display 13 and provides the progression prediction result 18 for viewing by the doctor. Note that the progression prediction result 18 is an example of a “prediction result” according to the technology of the present disclosure.

As shown in FIG. 2 as an example, in the present embodiment, the target input data 16 is data at the current point in time of the subject. The target input data 16 includes subject data 20, test data 21, and diagnostic data 22. The subject data 20 is data indicating attributes of a subject, and includes an age 23 and a gender 24 of the subject. Note that the current point in time is, for example, the same date as a transmission date of the prediction request 15. The transmission date of the prediction request 15 and a period from three days to one week before the transmission date may be included in the current point in time.

The test data 21 is data indicating a result of a test related to dementia of the subject, and includes a cognitive ability test score 25 which is cognitive function test data, a cerebrospinal fluid test result 26 which is blood/cerebrospinal fluid test data, a genetic test result 27 which is a genetic test data, and the MRI image 28 which is a morphological image test data. The cognitive ability test score 25 is, for example, a CDR-SOB score. The CSF test result 26 is, for example, the amount of phosphorylated tau protein (p-tau) 181 in CSF.

The genetic test result 27 is, for example, a test result of a genotype of the ApoE gene. The genotype of the ApoE gene is a combination of two types among three types of ApoE genes of ε2, ε3, and ε4 (ε2 and ε3, ε3 and ε4, and the like). A risk of developing Alzheimer's dementia in a person with a genotype including one or two of ε4 (ε2 and ε4, ε4 and ε4, and the like) is estimated to be about 3 to 12 times higher than that in a person with a genotype without ε4 (ε2 and ε3, ε3 and ε3, and the like).

The diagnostic data 22 is data indicating a result of diagnosis related to dementia of the subject, which has been made by a doctor at the current point in time with reference to the test data 21 and the like. The diagnostic data 22 is any one of normal control (NC), preclinical AD (PAD), mild cognitive impairment (MCI), and Alzheimer's dementia (ADM). In this way, there are a plurality of types of target input data 16, and the dementia progression prediction model 41 is a so-called multimodal machine learning model.

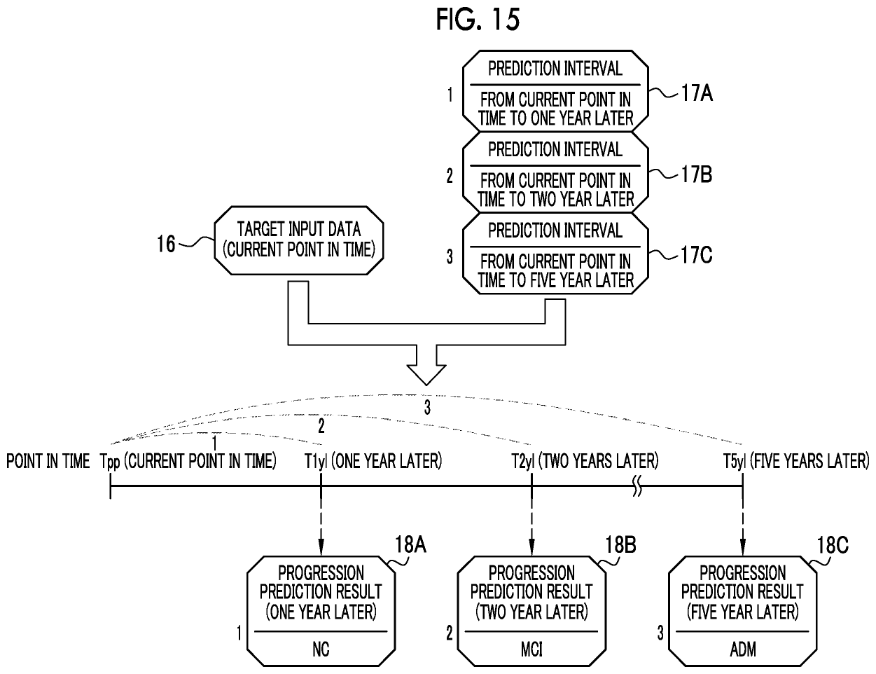

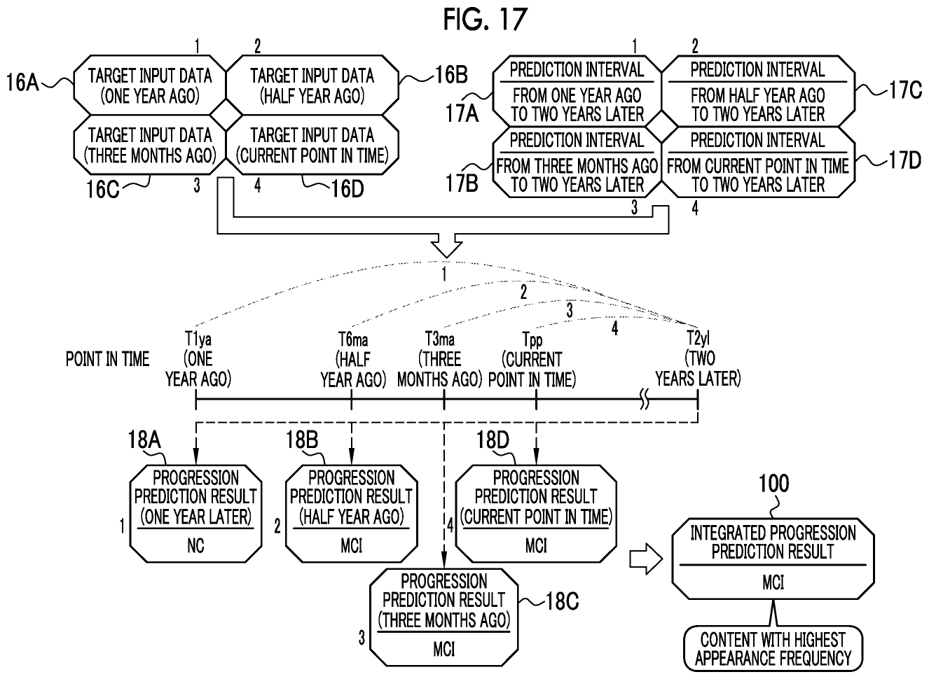

As shown in FIG. 3 as an example, in the present embodiment, the prediction interval 17 is an interval from the current point in time to the future point in time. The future point in time is preferably four years after the current point in time, more preferably three years after the current point in time, still more preferably two years after the current point in time, and even more preferably 18 months after the current point in time. The current point in time is an example of a “reference point in time” according to the technology of the present disclosure. In FIG. 3, two years later is an example of a “future point in time” according to the technology of the present disclosure. The prediction interval 17 in this case is two years. Note that the expression of a time interval, such as “two years later”, is merely an expression based on the current point in time. The same applies to subsequent “one year later”, “five years later” (both refer to FIG. 15), “half a year ago”, “three months ago” (both refer to FIG. 17), and the like.

In clinical trials, the drug efficacy is evaluated in a predetermined period (for example, two years, 18 months). Therefore, in a case where the technology of the present disclosure is used for predicting drug efficacy in clinical trials, it is possible to select a subject who progresses to dementia or MCI during the period of the clinical trials, and it is possible to perform an appropriate drug efficacy evaluation. In addition, it is possible to start treatment at an early stage for a subject who progresses to dementia or MCI at an early stage from the current point in time, and it is possible to improve the therapeutic effect.

As shown in FIG. 4 as an example, as in the diagnostic data 22, the progression prediction result 18 is a content indicating whether the subject is normal control, preclinical AD, mild cognitive impairment, or Alzheimer's dementia.

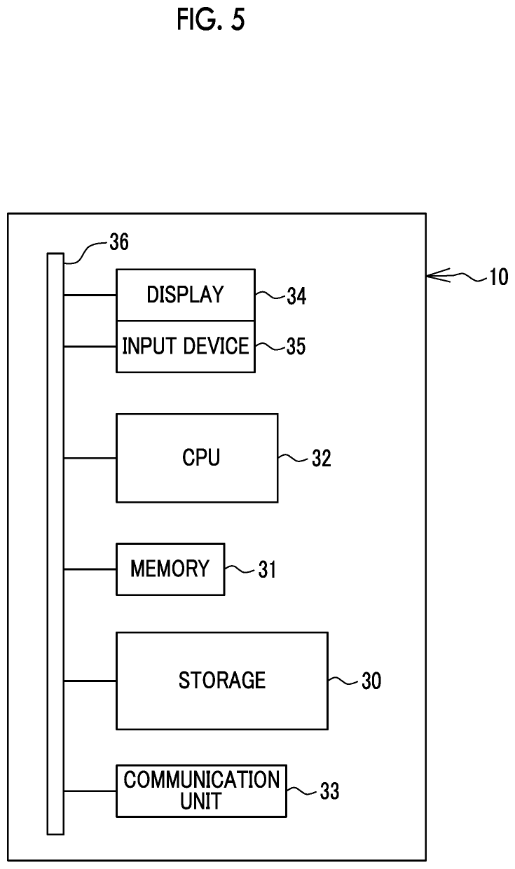

As shown in FIG. 5 as an example, a computer constituting the dementia progression prediction server 10 comprises a storage 30, a memory 31, a central processing unit (CPU) 32, a communication unit 33, a display 34, and an input device 35. These components are connected to each other through a bus line 36. Note that CPU 32 is an example of a “processor” according to the technology of the present disclosure.

The storage 30 is a hard disk drive built in the computer constituting the dementia progression prediction server 10 or connected via a cable or a network. Alternatively, the storage 30 is a disk array in which a plurality of hard disk drives are connected in series. The storage 30 stores a control program such as an operating system, various application programs, various types of data associated with these programs, and the like. A solid state drive may be used instead of the hard disk drive.

The memory 31 is a work memory for the CPU 32 to execute processing. The CPU 32 loads the program stored in the storage 30 into the memory 31 and executes processing corresponding to the program. Thus, the CPU 32 integrally controls the respective units of the computer. The memory 31 may be built in the CPU 32.

The communication unit 33 controls transmission of various types of information to and from an external device such as the user terminal 11. The display 34 displays various screens. Various screens have operation functions by a graphical user interface (GUI). The computer constituting the dementia progression prediction server 10 receives inputs of operation instructions from the input device 35 through various screens. The input device 35 is a keyboard, a mouse, a touch panel, a microphone for voice input, or the like.

As shown in FIG. 6 as an example, an operation program 40 is stored in the storage 30 of the dementia progression prediction server 10. The operation program 40 is an application program for causing the computer to function as the dementia progression prediction server 10. That is, the operation program 40 is an example of an “operation program of a medical support device” according to the technology of the present disclosure. The storage 30 also stores a dementia progression prediction model 41. The dementia progression prediction model 41 is an example of a “machine learning model” according to the technology of the present disclosure.

In a case where the operation program 40 is activated, the CPU 32 of the computer constituting the dementia progression prediction server 10 cooperates with the memory 31 and the like to function as a reception unit 45, a read and write (hereinafter abbreviated as RW) control unit 46, a prediction unit 47, and a distribution control unit 48.

The reception unit 45 receives the prediction request 15 from the user terminal 11. Since the prediction request 15 includes the target input data 16 and the prediction interval 17 as described above, the reception unit 45 receives the prediction request 15 to acquire the target input data 16 and the prediction interval 17. The reception unit 45 outputs the acquired target input data 16 and prediction interval 17 to the prediction unit 47. Furthermore, the reception unit 45 outputs a terminal ID of the user terminal 11 (not shown) to the distribution control unit 48.

The RW control unit 46 controls storage of various types of data in the storage 30 and reading out of various types of data in the storage 30. For example, the RW control unit 46 reads out the dementia progression prediction model 41 from the storage 30 and outputs the dementia progression prediction model 41 to the prediction unit 47.

The prediction unit 47 inputs the target input data 16 and the prediction interval 17 to the dementia progression prediction model 41, and causes the dementia progression prediction model 41 to output the progression prediction result 18. The prediction unit 47 outputs the progression prediction result 18 to the distribution control unit 48.

The distribution control unit 48 performs control to distribute the progression prediction result 18 to the user terminal 11 that is a transmission source of the prediction request 15. In this case, the distribution control unit 48 specifies the user terminal 11 that is the transmission source of the prediction request 15 based on the terminal ID from the reception unit 45.

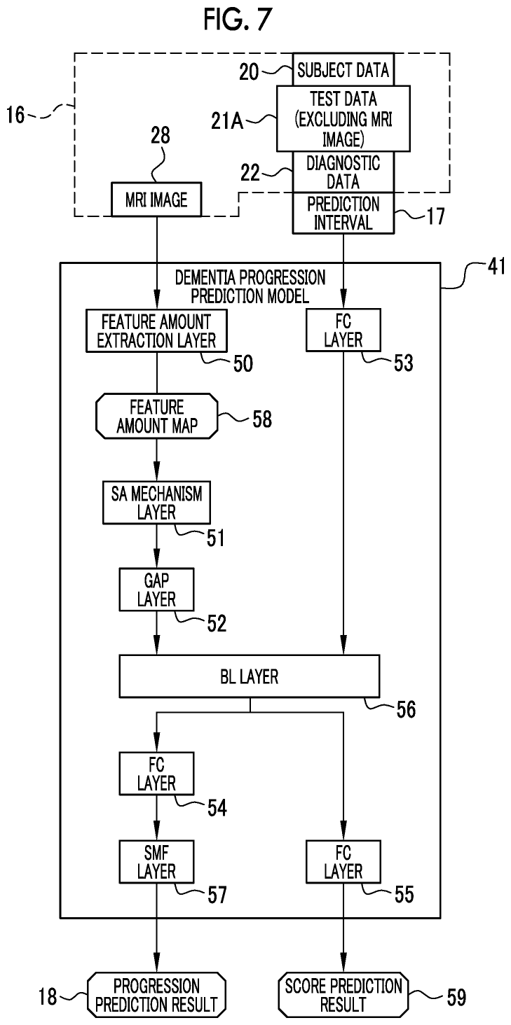

As shown in FIG. 7 as an example, the dementia progression prediction model 41 includes a feature amount extraction layer 50, a self-attention (hereinafter abbreviated as SA) mechanism layer 51, a global average pooling (hereinafter abbreviated as GAP) layer 52, fully connected (hereinafter abbreviated as FC) layers 53, 54, and 55, a bi-linear (hereinafter abbreviated as BL) layer 56, and a softmax function (hereinafter abbreviated as SMF) layer 57.

The feature amount extraction layer 50 is, for example, densely connected convolutional networks (DenseNet). The MRI image 28 is input to the feature amount extraction layer 50. The feature amount extraction layer 50 performs convolution processing or the like on the MRI image 28 to convert the MRI image 28 into a feature amount map 58. The feature amount extraction layer 50 outputs the feature amount map 58 to the SA mechanism layer 51.

The SA mechanism layer 51 performs convolution processing on the feature amount map 58 while changing the coefficients of a convolution filter according to the feature amount of the feature amount map 58 to be processed. The convolution processing performed by the SA mechanism layer 51 is hereinafter referred to as SA convolution processing. The SA mechanism layer 51 outputs the feature amount map 58 after the SA convolution processing to the GAP layer 52.

The GAP layer 52 performs global average pooling processing on the feature amount map 58 after the SA convolution processing. The global average pooling processing is processing of obtaining an average value of feature amounts for each channel of the feature amount map 58. For example, in a case where the number of channels of the feature amount map 58 is 512, an average value of 512 feature amounts is obtained by the global average pooling processing. The GAP layer 52 outputs the obtained average value of the feature amounts to the BL layer 56.

The subject data 20, test data 21A excluding the MRI image 28, the diagnostic data 22, and the prediction interval 17 are input to the FC layer 53. The gender 24 of the subject data 20 is input as a numerical value such as 1 for male and 0 for female. Similarly, the genetic test result 27 of the test data 21 is input as a numerical value such as 1 for the combination of ε2 and ε3 and 2 for the combination of ε3 and ε3. The diagnostic data 22 is similarly input as a numerical value. The FC layer 53 has an input layer including units corresponding to the number of data items and an output layer including units corresponding to the number of data items handled by the BL layer 56. Each unit of the input layer and each unit of the output layer are fully connected to each other, and weights are set for each unit. The subject data 20, the test data 21A excluding the MRI image 28, the diagnostic data 22, and the prediction interval 17 are input to each unit of the input layer. The product sum of the each piece of the data and the weight which is set for each unit is an output value of each unit of the output layer. The FC layer 53 outputs the output value of the output layer to the BL layer 56.

The BL layer 56 performs bi-linear processing on the average value of the feature amounts from the GAP layer 52 and the output value from the FC layer 53. The BL layer 56 outputs the values after the bi-linear processing to the FC layers 54 and 55. For the BL layer 56 and the bi-linear processing, the following document can be referred to.

<Goto, T. etc, Multi-modal deep learning for predicting progression of Alzheimer's disease using bi-linear shake fusion, Proc. SPIE 11314, Medical Imaging (2020)>

The FC layer 54 converts the values after the bi-linear processing into variables handled by the SMF of the SMF layer 57. Similarly to the FC layer 53, the FC layer 54 has an input layer including units corresponding to the number of values after the bi-linear processing and an output layer including units corresponding to the number of variables handled by the SMF. Each unit of the input layer and each unit of the output layer are fully connected to each other, and weights are set for each unit. A value after the bi-linear processing is input to each unit of the input layer. The product sum of the value after the bi-linear processing and the weight which is set for each unit is an output value of each unit of the output layer. This output value is a variable handled by the SMF. The FC layer 54 outputs variables handled by the SMF to the SMF layer 57. The SMF layer 57 outputs the progression prediction result 18 by applying the variables to the SMF.

The FC layer 55 converts the values after the bi-linear processing into a score prediction result 59. Similarly to the FC layers 53 and 54, the FC layer 55 has an input layer including units corresponding to the number of values after the bi-linear processing, and an output layer of the score prediction result 59. Each unit of the input layer and the output layer are fully connected to each other, and weights are set for each. A value after the bi-linear processing is input to each unit of the input layer. The product sum of the value after the bi-linear processing and the weight which is set for each unit is an output value of the output layer. This output value is the score prediction result 59. The score prediction result 59 is a prediction result of the score itself of the cognitive ability test of the subject, here the CDR-SOB score itself, at the future point in time designated by the prediction interval 17. The CDR-SOB score takes a value of 0 to 18, where 0 is normal control and 18 is the maximum cognitive impairment. In this way, the dementia progression prediction model 41 is a so-called multi-task machine learning model that outputs the progression prediction result 18 and the score prediction result 59.

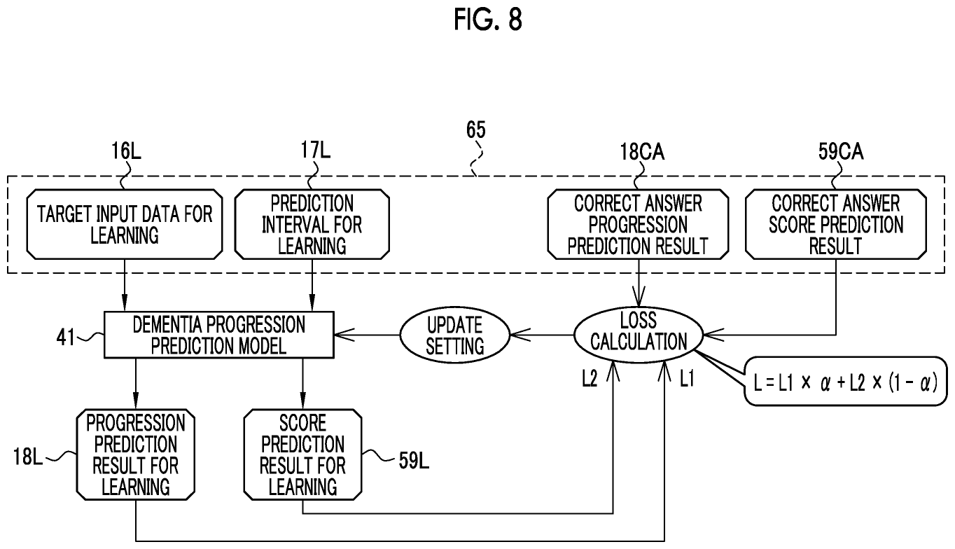

As shown in FIG. 8 as an example, the dementia progression prediction model 41 is trained by being giving supervised training data (also referred to as training data or learning data) 65 in a learning phase. The supervised training data 65 is a set of target input data for learning 16L, a prediction interval for learning 17L, a correct answer progression prediction result 18CA, and a correct answer score prediction result 59CA. The target input data for learning 16L is, for example, the target input data 16 of a certain sample subject (including a patient, the same applies hereinafter) accumulated in a database such as ADNI at a first point in time. The prediction interval for learning 17L is an interval from the first point in time to a second point in time in the future after the first point in time.

The correct answer progression prediction result 18CA is a diagnosis result of dementia that is actually given to the sample subject by the doctor at the second point in time. The correct answer score prediction result 59CA is a score of a cognitive ability test that is actually performed by the sample subject at the second point in time. The target input data for learning 16L is an example of “accumulated input data related to dementia at two or more points in time” according to the technology of the present disclosure. Further, the prediction interval for learning 17L is an example of a “time interval of input data” according to the technology of the present disclosure.

In the learning phase, the target input data for learning 16L and the prediction interval for learning 17L are input to the dementia progression prediction model 41. The dementia progression prediction model 41 outputs a progression prediction result for learning 18L and a score prediction result for learning 59L for the target input data for learning 16L and the prediction interval for learning 17L.

A loss calculation of the dementia progression prediction model 41 using a cross-entropy function is performed based on the progression prediction result for learning 18L and the correct answer progression prediction result 18CA. A result of the loss calculation is hereinafter referred to as a loss L1. In addition, a loss calculation of the dementia progression prediction model 41 using a regression loss function such as a mean squared error is performed based on the score prediction result for learning 59L and the correct answer score prediction result 59CA. A result of the loss calculation is hereinafter referred to as a loss L2.

Various coefficients of the dementia progression prediction model 41 are set to be updated according to the losses L1 and L2, and the dementia progression prediction model 41 is updated according to the update settings. The update setting is performed based on a total loss L represented by Equation (1) below. Note that α is a weight.

L=L1×α+L2×(1−α) (1)

That is, the total loss L is a weighted sum of the loss L1 and the loss L2. α is, for example, 0.5.

In the learning phase, the series of processes of an input of the target input data for learning 16L and the prediction interval for learning 17L to the dementia progression prediction model 41, an output of the progression prediction result for learning 18L and score prediction result for learning 59L from the dementia progression prediction model 41, a loss calculation, an update setting, and an update of the dementia progression prediction model 41 are repeatedly performed while the supervised training data 65 is exchanged at least twice. The repetition of the series of processes is ended in a case where the prediction accuracy of the progression prediction result for learning 18L and the score prediction result for learning 59L with respect to the correct answer progression prediction result 18CA and the correct answer score prediction result 59CA reaches a predetermined set level. The dementia progression prediction model 41 of which the prediction accuracy reaches the set level in this way is stored in the storage 30, and is used in the prediction unit 47. The learning may be ended in a case where the series of processes is repeated a set number of times, regardless of the prediction accuracy of the progression prediction result for learning 18L and the score prediction result for learning 59L with respect to the correct answer progression prediction result 18CA and the correct answer score prediction result 59CA.

Note that, although 0.5 has been described as an example of α, the technology of the present disclosure is not limited thereto. Also, α is not limited to a fixed value, and a may be changed, for example, between the initial period of the learning phase and the other period. For example, in the initial period of the learning phase, α is set to 1, and as the learning progresses, α is gradually decreased, and is eventually set to a fixed value, for example, 0.5.

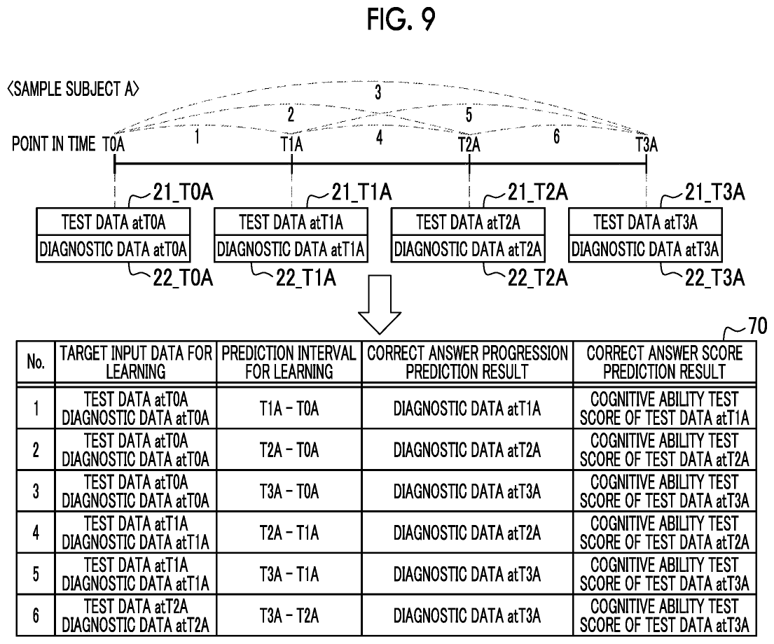

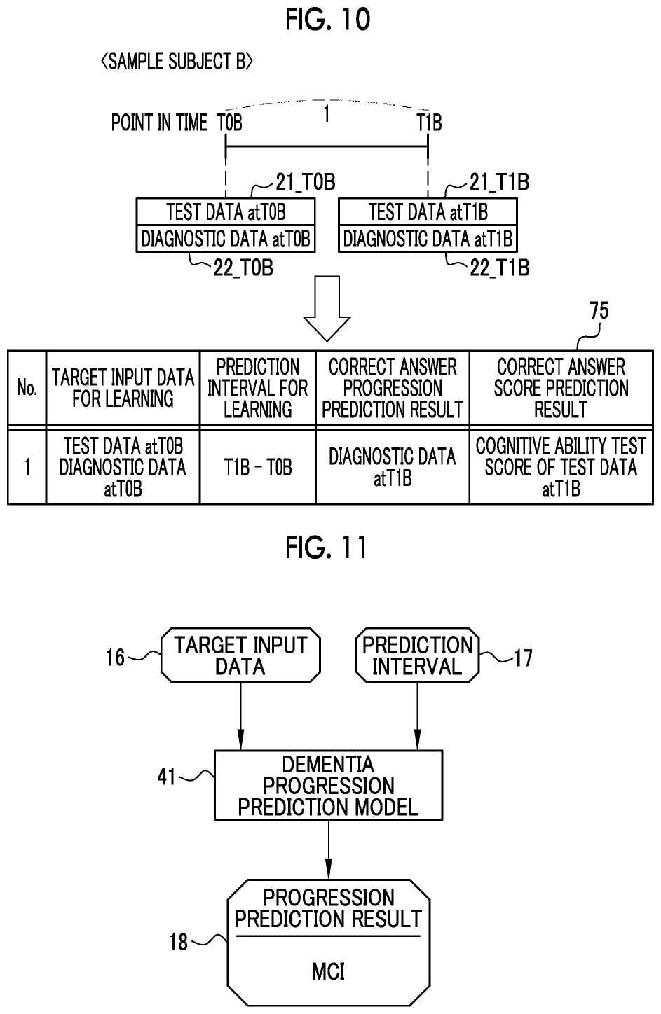

FIGS. 9 and 10 are diagrams for describing the formation of the supervised training data 65. FIG. 9 shows a case of a sample subject A. FIG. 10 shows a case of a sample subject B.

In FIG. 9, the sample subject A has test data 21 and diagnostic data 22 at four points in time of points in time T0A, T1A, T2A, and T3A. Specifically, the sample subject A has test data 21_T0A (denoted as test data atT0A in FIG. 9) and diagnostic data 22_T0A (denoted as diagnostic data atT0A in FIG. 9) at a point in time T0A, test data 21_T1A (denoted as test data atT1A in FIG. 9) and diagnostic data 22_T1A (denoted as diagnostic data atT1A in FIG. 9) at a point in time T1A, test data 21_T2A (denoted as test data atT2A in FIG. 9) and diagnostic data 22_T2A (denoted as diagnostic data atT2A in FIG. 9) at a point in time T2A, and test data 21_T3A (denoted as test data atT3A in FIG. 9) and diagnostic data 22_T3A (denoted as diagnostic data atT3A in FIG. 9) at a point in time T3A.

In this case, as shown in Table 70, it is possible to generate six pieces of supervised training data 65 of No. 1 to No. 6. For example, the supervised training data 65 of No. 1 is data related to the point in time T0A and the point in time T1A. The target input data for learning 16L is the test data 21_T0A and the diagnostic data 22_T0A at the point in time T0A. The prediction interval for learning 17L is a difference (T1A-T0A) between the point in time T0A and the point in time T1A. The correct answer progression prediction result 18CA is the diagnostic data 22_T1A at the point in time T1A. The correct answer score prediction result 59CA is the cognitive ability test score 25 of the test data 21_T1A at the point in time T1A. In this case, the point in time T0A corresponds to the above-mentioned first point in time, and the point in time T1A corresponds to the above-mentioned second point in time.

In addition, for example, the supervised training data 65 of No. 4 is data related to the point in time T1A and the point in time T2A. The target input data for learning 16L is the test data 21_T1A and the diagnostic data 22_T1A at the point in time T1A. The prediction interval for learning 17L is a difference (T2A-T1A) between the point in time T1A and the point in time T2A. The correct answer progression prediction result 18CA is the diagnostic data 22_T2A at the point in time T2A. The correct answer score prediction result 59CA is the cognitive ability test score 25 of the test data 21_T2A at the point in time T2A. In this case, the point in time T1A corresponds to the above-mentioned first point in time, and the point in time T2A corresponds to the above-mentioned second point in time. Note that the numbers of No. 1 to No. 6 correspond to the numbers 1 to 6 of the arcs connecting the points in time on the time axis. The same applies to subsequent FIG. 10 and the like.

In FIG. 10, the sample subject B has test data 21 and diagnostic data 22 at two points in time of points in time T0B and T1B. Specifically, the sample subject B has test data 21_T0B (denoted as test data atT0B in FIG. 10) and diagnostic data 22_T0B (denoted as diagnostic data atT0B in FIG. 10) at a point in time T0B and test data 21_T1B (denoted as test data atT1B in FIG. 10) and diagnostic data 22_T1B (denoted as diagnostic data atT1B in FIG. 10) at a point in time T1B.

In this case, as shown in Table 75, it is possible to generate one piece of supervised training data 65 of No. 1. That is, the supervised training data 65 of No. 1 is data related to the point in time T0B and the point in time T1B. The target input data for learning 16L is the test data 21_T0B and the diagnostic data 22_T0B at the point in time T0B. The prediction interval for learning 17L is a difference (T1B−T0B) between the point in time T0B and the point in time T1B. The correct answer progression prediction result 18CA is the diagnostic data 22_T1B at the point in time T1B. The correct answer score prediction result 59CA is the cognitive ability test score 25 of the test data 21_T1B at the point in time T1B. In this case, the point in time T0B corresponds to the above-mentioned first point in time, and the point in time T1B corresponds to the above-mentioned second point in time. In this way, the supervised training data 65 includes the test data 21 and the diagnostic data 22 at two points in time and the interval between the two points in time out of the test data 21 and the diagnostic data 22 at two or more points in time of the same sample subject. Although not shown, for example, in a case of a sample subject having the test data 21 and the diagnostic data 22 at six points in time, ₆C₂=6×5÷2=15, and it is thus possible to generate 15 pieces of supervised training data 65. Further, for example, in a case of a sample subject having the test data 21 and the diagnostic data 22 at eight points in time, ₈C₂=8×7÷2=28, and it is thus possible to generate 28 pieces of supervised training data 65.

The supervised training data 65 is not limited to data including the input data related to dementia at two or more points in time of the same sample subject and the time intervals thereof. The input data related to dementia of a plurality of sample subjects having the same and/or similar dementia symptoms and time intervals thereof may be combined to generate the input data related to dementia at two or more points in time and time intervals thereof, which may be used as the supervised training data 65. Examples of the sample subject having the same and/or similar dementia symptoms include the sample subject having the same and/or similar test data 21 and/or the diagnostic data 22. In addition, the input data related to dementia of a plurality of sample subjects having the same and/or similar attributes and time intervals thereof may be combined to generate the input data related to dementia at two or more points in time and time intervals thereof, which may be used as the supervised training data 65. Examples of the sample subject having the same and/or similar attributes include the sample subject having the same and/or similar age 23 and/or the gender 24. The input data related to dementia of a plurality of sample subjects having the same and/or similar dementia symptoms and having the same and/or similar attributes and time intervals thereof may be combined to generate the input data related to dementia at two or more points in time and time intervals thereof, which may be used as the supervised training data 65.

As shown in FIG. 11 as an example, the prediction unit 47 inputs the target input data 16 and the prediction interval 17 to the dementia progression prediction model 41, and causes the dementia progression prediction model 41 to output the progression prediction result 18. The score prediction result 59 is also output from the dementia progression prediction model 41, but the prediction unit 47 discards the score prediction result 59 and outputs only the progression prediction result 18 to the distribution control unit 48. FIG. 11 exemplifies a case where the progression prediction result 18 is mild cognitive impairment.

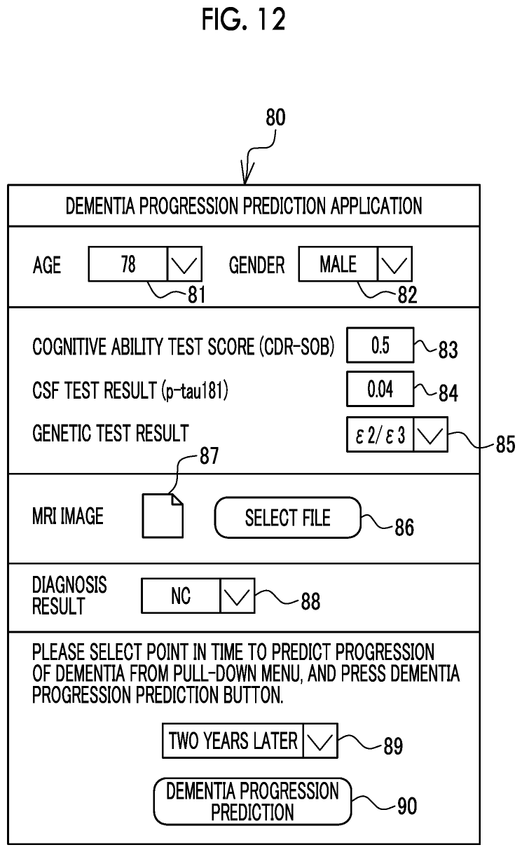

FIG. 12 shows an example of a dementia progression prediction screen 80 displayed on the display 13 of the user terminal 11. On the dementia progression prediction screen 80, a pull-down menu 81 for selecting the age 23, a pull-down menu 82 for selecting the gender 24, an input box 83 for the cognitive ability test score 25, an input box 84 for the CSF test result 26, and a pull-down menu 85 for selecting the genetic test result 27 are provided.

On the dementia progression prediction screen 80, a file selection button 86 for selecting a file of the MRI image 28 is provided. In a case where the file of the MRI image 28 is selected, a file icon 87 is displayed next to the file selection button 86. The file icon 87 is not displayed in a case where a file is not selected. Further, a pull-down menu 88 for selecting the diagnosis result (diagnostic data 22) is provided on the dementia progression prediction screen 80.

A pull-down menu 89 for selecting the prediction interval 17 and a dementia progression prediction button 90 are disposed at the bottom of the dementia progression prediction screen 80. In a case where the desired prediction interval 17 is selected in the pull-down menu 89 and the dementia progression prediction button 90 is further selected, the prediction request 15 including the target input data 16 and the prediction interval 17 is transmitted from the user terminal 11 to the dementia progression prediction server 10. The target input data 16 is composed of the contents selected by the pull-down menus 81, 82, 85, and 88, the contents input in the input boxes 83 and 84, and the MRI image 28 selected by the file selection button 86. The prediction interval 17 includes the contents selected in the pull-down menu 89.

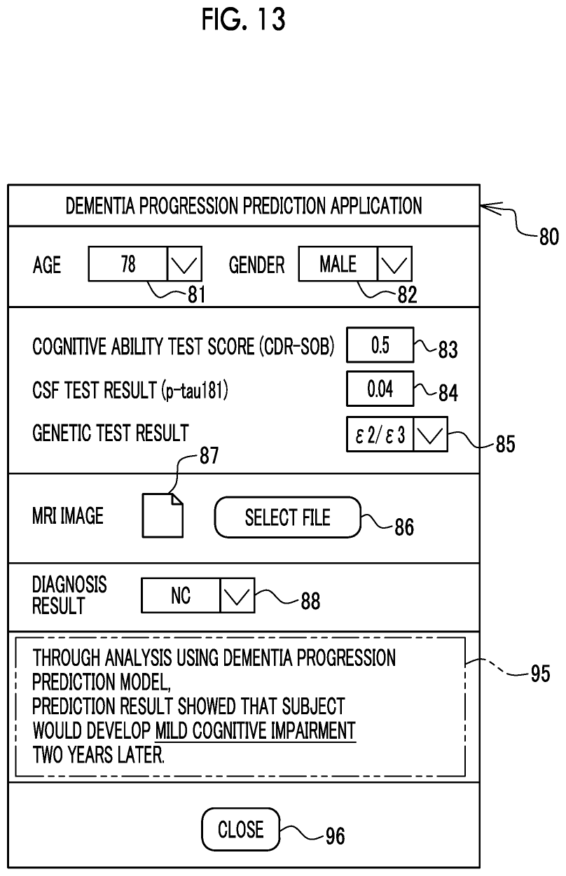

In a case where the progression prediction result 18 from the dementia progression prediction server 10 is received, the dementia progression prediction screen 80 transitions as shown in FIG. 13 as an example. Specifically, a message 95 indicating the progression prediction result 18 is displayed. FIG. 13 exemplifies a case where the progression prediction result 18 is mild cognitive impairment. The display of the dementia progression prediction screen 80 disappears by selecting a close button 96.

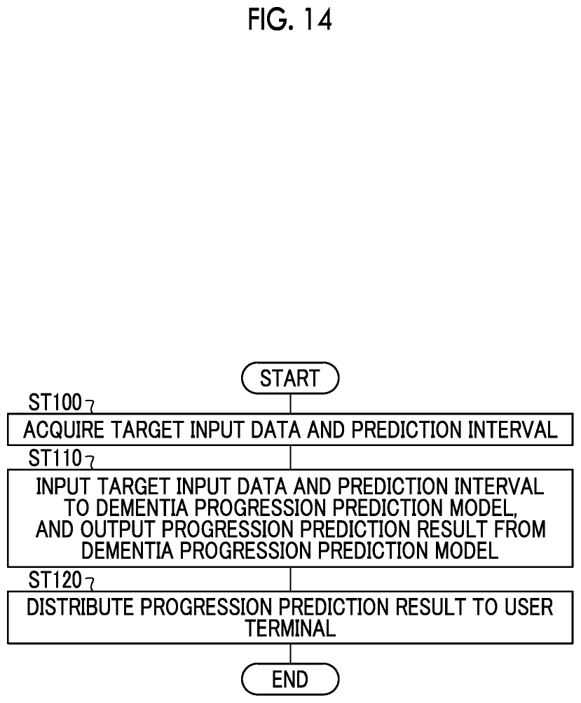

Next, an operation according to the above configuration will be described with reference to a flowchart shown in FIG. 14. First, in a case where the operation program 40 is activated in the dementia progression prediction server 10, as shown in FIG. 6, the CPU 32 of the dementia progression prediction server 10 functions as the reception unit 45, the RW control unit 46, the prediction unit 47, and the distribution control unit 48.

First, in the reception unit 45, the prediction request 15 from the user terminal 11 is received, and thus the target input data 16 and the prediction interval 17 are acquired (Step ST100). The target input data 16 and the prediction interval 17 are output from the reception unit 45 to the prediction unit 47.

As shown in FIG. 11, in the prediction unit 47, the target input data 16 and the prediction interval 17 are input to the dementia progression prediction model 41, and the progression prediction result 18 is output from the dementia progression prediction model 41 (Step ST110). The progression prediction result 18 is output from the prediction unit 47 to the distribution control unit 48 and is distributed to the user terminal 11 that is the transmission source of the prediction request 15 under the control of the distribution control unit 48 (Step ST120).

As described above, the CPU 32 of the dementia progression prediction server 10 comprises the reception unit 45, the prediction unit 47. By receiving the prediction request 15, the reception unit 45 acquires the target input data 16 which is input data related to dementia of a subject whose progression of dementia is to be predicted, and the prediction interval 17 which is an interval from a reference point in time to a future point in time at which prediction is performed. The prediction unit 47 inputs the target input data 16 and the prediction interval 17 to the dementia progression prediction model 41, and causes the dementia progression prediction model 41 to output the progression prediction result 18 which is the prediction result regarding dementia of the subject at the future point in time.

As shown in FIGS. 8 to 10, the dementia progression prediction model 41 is trained using the supervised training data 65 including the accumulated target input data for learning 16L related to dementia at two or more points in time and the prediction interval for learning 17L. Since the prediction interval for learning 17L is included as the time interval of the input data, it is possible to improve the prediction accuracy of the progression prediction result 18 as compared with the method of Document 1 in which test data of three or more points in time are provided as a set of supervised training data to the RNN for learning. Since more supervised training data 65 can be prepared than in the method of Document 1, overlearning can be prevented. Therefore, it is possible to suppress a decrease in accuracy of predicting the progression of dementia. As a result, it is possible to improve the accuracy of predicting the progression of dementia.

The input data includes the test data 21 indicating a result of a test related to the dementia and the diagnostic data 22 indicating a result of the diagnosis related to the dementia. Therefore, it is possible to contribute to improving the prediction accuracy of the progression prediction result 18. Note that the input data may include at least one of the test data 21 or the diagnostic data 22.

The target input data 16 is data of the subject at a current point in time, and a reference point in time is the current point in time. Therefore, the doctor can ascertain how the degree of progression of the subject's dementia will be from the current point in time based on the progression prediction result 18. Based on this, the doctor can propose an accurate treatment policy at the current point in time, such as positively administering a drug that suppresses the progression of dementia, and apply the treatment policy to the subject.

In addition, the prediction unit 47, the number of prediction intervals 17 to be input to the dementia progression prediction model 41 is not limited to one. As shown in FIG. 15 as an example, a plurality of prediction intervals 17 with a current point in time as a reference point in time may be input to the dementia progression prediction model 41, and a plurality of progression prediction results 18 may be output from the dementia progression prediction model 41.

FIG. 15 exemplifies a case where the prediction unit 47 inputs three prediction intervals 17A, 17B, and 17C to the dementia progression prediction model 41. The prediction interval 17A is an interval from the current point in time (denoted as Tpp in FIG. 15) to one year later (denoted as T1yl in FIG. 15), that is, one year. The prediction interval 17B is an interval from the current point in time to two years later (denoted as T2yl in FIG. 15), that is, two years. The prediction interval 17C is an interval from the current point in time to five years later (denoted as T5yl in FIG. 15), that is, five years. One year later, two years later, and five years later are examples of “future points in time” according to the technology of the present disclosure.

The dementia progression prediction model 41 outputs a progression prediction result 18A one year later with respect to the input of the target input data 16 and the prediction interval 17A at the current point in time. In addition, the dementia progression prediction model 41 outputs a progression prediction result 18B two years later with respect to the input of the target input data 16 and the prediction interval 17B at the current point in time. Further, the dementia progression prediction model 41 outputs a progression prediction result 18C five years later with respect to the input of the target input data 16 and the prediction interval 17C at the current point in time. FIG. 15 exemplifies a case where the progression prediction result 18A is normal control, the progression prediction result 18B is mild cognitive impairment, and the progression prediction result 18C is Alzheimer's dementia. In this case, although not shown, the messages 95 indicating the three progression prediction results 18A, 18B, and 18C are displayed side by side on the dementia progression prediction screen 80.

In this way, in a case where the plurality of prediction intervals 17 with the current point in time as the reference point in time are input to the dementia progression prediction model 41, and the plurality of progression prediction results 18 are output from the dementia progression prediction model 41, the doctor can ascertain the progression prediction results 18 at a plurality of future points in time at a glance. The doctor can understand the elapse of the degree of progression of dementia of the subject over time. For example, in FIG. 15, it can be seen that the progression prediction result 18 deteriorates with each year.

### Second Embodiment

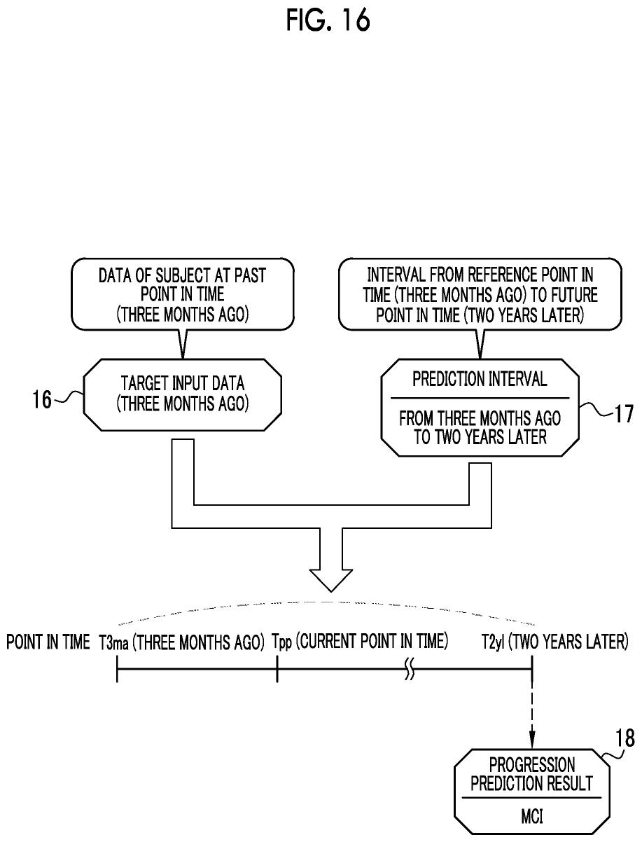

In the first embodiment, the current point in time is exemplified as the reference point in time, but the present disclosure is not limited thereto. As shown in FIG. 16 as an example, the reference point in time may be a past point in time.

FIG. 16 exemplifies a case where the prediction unit 47 inputs the target input data 16 three months ago (denoted as T3ma in FIG. 16) and the prediction interval 17 from three months ago to two years later, that is, the prediction interval 17 of two years and three months to the dementia progression prediction model 41. Three months ago is an example of a “past point in time” according to the technology of the present disclosure. In addition, two years later is an example of a “future point in time” according to the technology of the present disclosure. The dementia progression prediction model 41 outputs a progression prediction result 18 from the current point in time to two years later with respect to the input of the target input data 16 three months ago and the prediction interval 17 from three months ago to two years later.

In this way, in the second embodiment, the target input data 16 is data of the subject at the past point in time, and the reference point in time is the past point in time. Accordingly, the doctor can predict the progression of dementia of the subject using the target input data 16 at the past point in time even without the target input data 16 at the current point in time.

### Third Embodiment

As shown in FIG. 17 as an example, the prediction unit 47 may cause the dementia progression prediction model 41 to output a plurality of pieces of target input data 16 at the past point in time and the current point in time and a plurality of progression prediction results 18 for each of a plurality of prediction intervals 17 with the past point in time and the current point in time as reference points in time and derive an integrated progression prediction result 100 in which the plurality of progression prediction results 18 are integrated. The integrated progression prediction result 100 is an example of an “integrated prediction result” according to the technology of the present disclosure.

FIG. 17 exemplifies a case where the prediction unit 47 inputs four pieces of target input data 16A, 16B, 16C, and 16D and four prediction intervals 17A, 17B, 17C, and 17D to the dementia progression prediction model 41. The target input data 16A is data from one year ago (denoted as T1ya in FIG. 17), and the target input data 16B is data from half a year ago (denoted as T6ma in FIG. 17). The target input data 16C is data three months ago, and the target input data 16D is data at the current point in time. The prediction interval 17A is from one year ago to two years later, that is, three years, and the prediction interval 17B is from half a year ago to two years later, that is, two years and six months. The prediction interval 17C is from three months ago to two years later, that is, two years and three months, and the prediction interval 17D is from the current point in time to two years later, that is, two years. One year ago, half a year ago, and three months ago are examples of “past points in time” according to the technology of the present disclosure. In addition, two years later is an example of a “future point in time” according to the technology of the present disclosure.

The dementia progression prediction model 41 outputs a progression prediction result 18A from one year ago to two years later with respect to the input of the target input data 16A one year ago and the prediction interval 17A from one year ago to two years later. The dementia progression prediction model 41 outputs a progression prediction result 18B from half a year ago to two years later with respect to the input of the target input data 16B half a year ago and the prediction interval 17B from half a year ago to two years later. In addition, the dementia progression prediction model 41 outputs a progression prediction result 18C from the three months ago to two years later with respect to the input of the target input data 16C three months ago and the prediction interval 17C from three months ago to two years later. Further, the dementia progression prediction model 41 outputs a progression prediction result 18D from the current point in time to two years later with respect to the input of the target input data 16D at the current point in time and the prediction interval 17D from the current point in time to two years later.

The prediction unit 47 sets the content with the highest appearance frequency in the progression prediction results 18A to 18D as the integrated progression prediction result 100. In FIG. 17, since only the progression prediction result 18A is normal control and all of the progression prediction results 18B to 18D are mild cognitive impairment, the prediction unit 47 sets mild cognitive impairment with the highest appearance frequency as the integrated progression prediction result 100. In this case, although not shown, a message indicating the integrated progression prediction result 100 is displayed on the dementia progression prediction screen 80. In a case where the content with the highest appearance frequency cannot be specified, such as a case where the number of normal control cases and the number of mild cognitive impairment cases are the same, for example, a case where the symptom is severer is set as the integrated progression prediction result 100.

In this way, in the third embodiment, in a case where the plurality of pieces of target input data 16 and the plurality of prediction intervals 17 corresponding to the plurality of reference points in time are acquired at the reception unit 45, the prediction unit 47 causes the dementia progression prediction model 41 to output the plurality of progression prediction results 18 for each of the plurality of pieces of target input data 16 and the plurality of prediction intervals 17 and derives the integrated progression prediction result 100 in which the plurality of progression prediction results 18 are integrated. Therefore, it is possible to improve the accuracy of predicting the progression of dementia as compared with a case where the progression of dementia is to be predicted using only the target input data 16 at one point in time. For example, in the case of FIG. 17, the progression prediction result 18A obtained based on only the target input data 16A one year ago is normal control, but according to the progression prediction results 18B to 18D based on the target input data 16B to 16D half a year ago, three months ago, and at the current point in time, mild cognitive impairment can be said to be a more reliable result.

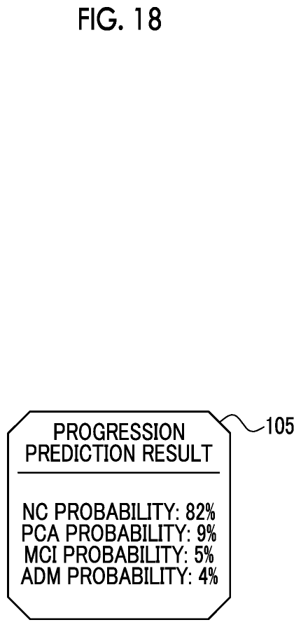

The progression prediction result is not limited to the progression prediction result 18 having a content of any one of normal control, preclinical AD, mild cognitive impairment, or Alzheimer's dementia as exemplified. As in a progression prediction result 105 shown in FIG. 18 as an example, a probability of each of normal control, preclinical AD, mild cognitive impairment, and Alzheimer's dementia may be used.

### Fourth Embodiment

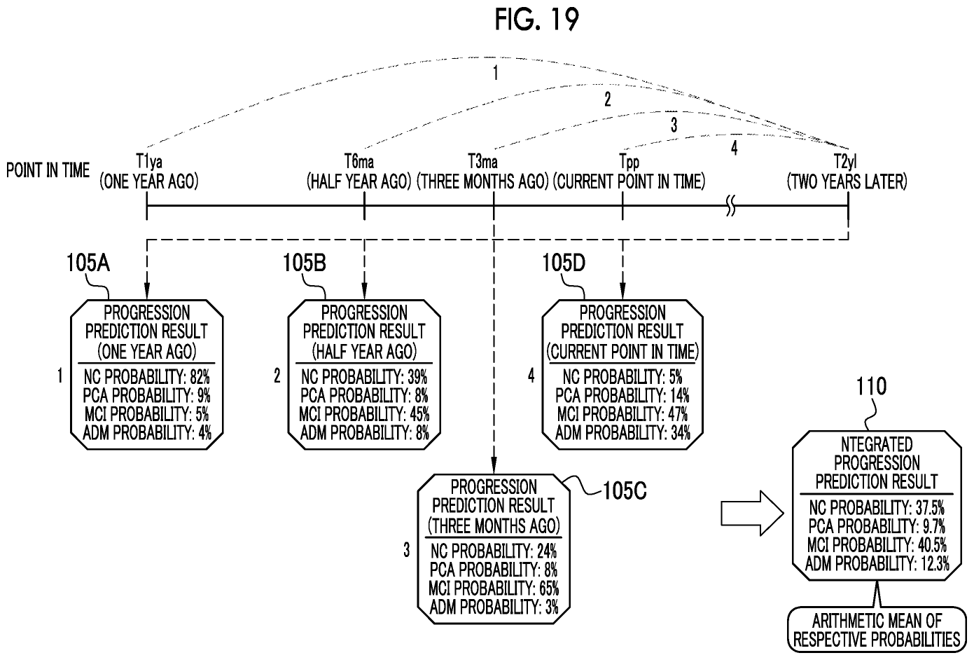

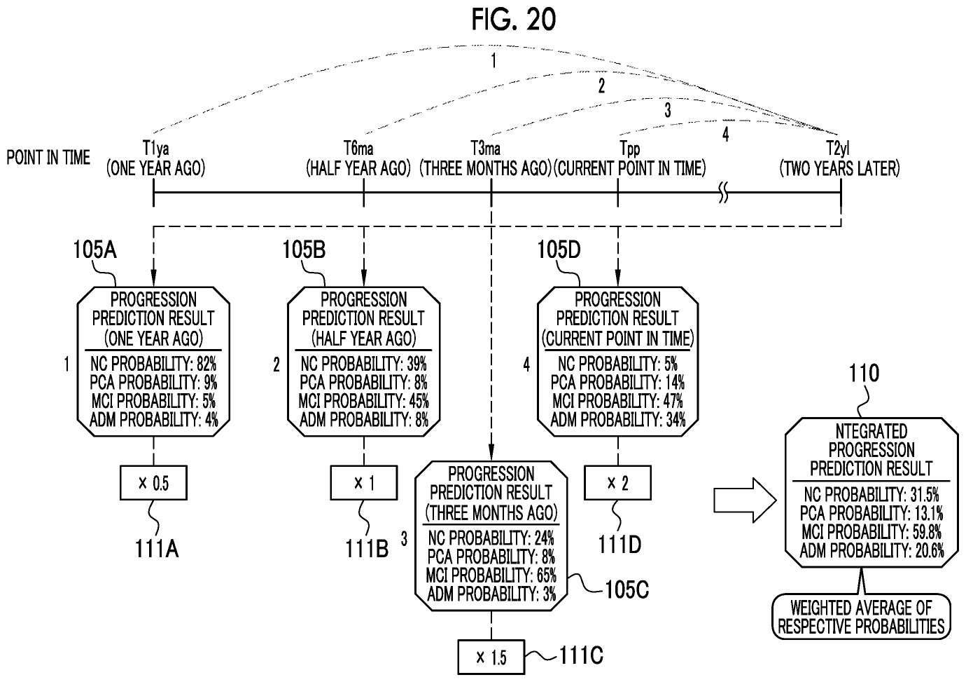

In the case of the progression prediction result 105, a method shown in FIG. 19 or FIG. 20 can be employed as an example of a method of deriving an integrated progression prediction result 110 in a case where a plurality of progression prediction results 105 are output from the dementia progression prediction model 41.

In FIGS. 19 and 20, a progression prediction result 105A is output from the dementia progression prediction model 41 with respect to the input of the target input data 16 one year ago and the prediction interval 17 from one year ago to two years later, which are not shown. A progression prediction result 105B is output from the dementia progression prediction model 41 with respect to the input of the target input data 16 half a year ago and the prediction interval 17 from half a year ago to two years later, which are not shown. A progression prediction result 105C is output from the dementia progression prediction model 41 with respect to the input of the target input data 16 three months ago and the prediction interval 17 from three months ago to two years later, which are not shown. A progression prediction result 105D is output from the dementia progression prediction model 41 with respect to the input of the target input data 16 at the current point in time and the prediction interval 17 from the current point in time to two years later, which are not shown.

In FIG. 19, the prediction unit 47 sets the arithmetic mean of the respective probabilities of the progression prediction results 105A to 105D as the integrated progression prediction result 110. For example, the probability of mild cognitive impairment in the integrated progression prediction result 110 will be considered. The probability of mild cognitive impairment is 5% for the progression prediction result 105A, 45% for the progression prediction result 105B, 65% for the progression prediction result 105C, and 47% for the progression prediction result 105D. Therefore, their arithmetic mean is (5+45+65+47)/4=40.5%.

In FIG. 20, the prediction unit 47 sets the weighted average of the respective probabilities of the progression prediction results 105A to 105D as the integrated progression prediction result 110. For the progression prediction result 105A, 0.5 is set as a weight 111A in a case where the weighted average is calculated, and for the progression prediction result 105B, 1 is set as a weight 111B. For the progression prediction result 105C, 1.5 is set as a weight 111C, and for the progression prediction result 105D, 2 is set as a weight 111D. Therefore, for example, in a case where the probability of Alzheimer's dementia in the integrated progression prediction result 110 is considered, the probability of Alzheimer's dementia is 4% for the progression prediction result 105A, 8% for the progression prediction result 105B, 3% for the progression prediction result 105C, and 34% for the progression prediction result 105D. Therefore, their weighted average is (4×0.5+8×1+3×1.5+34×2)/4≈20.6%. In the case of FIGS. 19 and 20, although not shown, a message indicating the integrated progression prediction result 110 is displayed on the dementia progression prediction screen 80.

In this way, the prediction unit 47 changes the weights 111A to 111D given to the progression prediction results 105A to 105D in a case where the weighted average is calculated according to the prediction interval 17. More specifically, the prediction unit 47 sets the weight 111D given to the progression prediction result 105D at the current point in time to 2, which is the maximum, and sets the value of the weight 111 smaller as the reference point in time of the prediction interval 17 moves away from the current point in time.

In this way, the prediction unit 47 derives the arithmetic mean of the plurality of progression prediction results 105 as the integrated progression prediction result 110. Alternatively, the prediction unit 47 derives the weighted average of the plurality of progression prediction results 105 as the integrated progression prediction result 110. Therefore, as in the case of the third embodiment, it is possible to improve the accuracy of predicting the progression of dementia as compared with a case where the progression of dementia is to be predicted using only the target input data 16 at one point in time. According to the arithmetic mean, it is possible to easily derive the integrated progression prediction result 110. In contrast, according to the weighted average, it is possible to derive the integrated progression prediction result 110 in which the importance as the data of the plurality of progression prediction results 105 is considered.

The prediction accuracy of the progression prediction result 105 changes depending on whether the prediction interval 17 is long or short. Specifically, in a case where the prediction interval 17 is relatively long, the prediction accuracy of the progression prediction result 105 tends to be lower than in a case where the prediction interval 17 is relatively short. Therefore, as shown in FIG. 20, in a case where the value of the weight 111 is set smaller as the reference point in time of the prediction interval 17 moves away from the current point in time, the progression prediction results 105 (progression prediction result 105A one year ago and progression prediction result 105B half a year ago) with relatively low prediction accuracy are less likely to be reflected in the integrated progression prediction result 110. In other words, the progression prediction results 105 (progression prediction result 105C three months ago and progression prediction result 105D at the current point in time) with relatively high prediction accuracy are likely to be reflected in the integrated progression prediction result 110. Therefore, a more reliable integrated progression prediction result 110 can be derived.

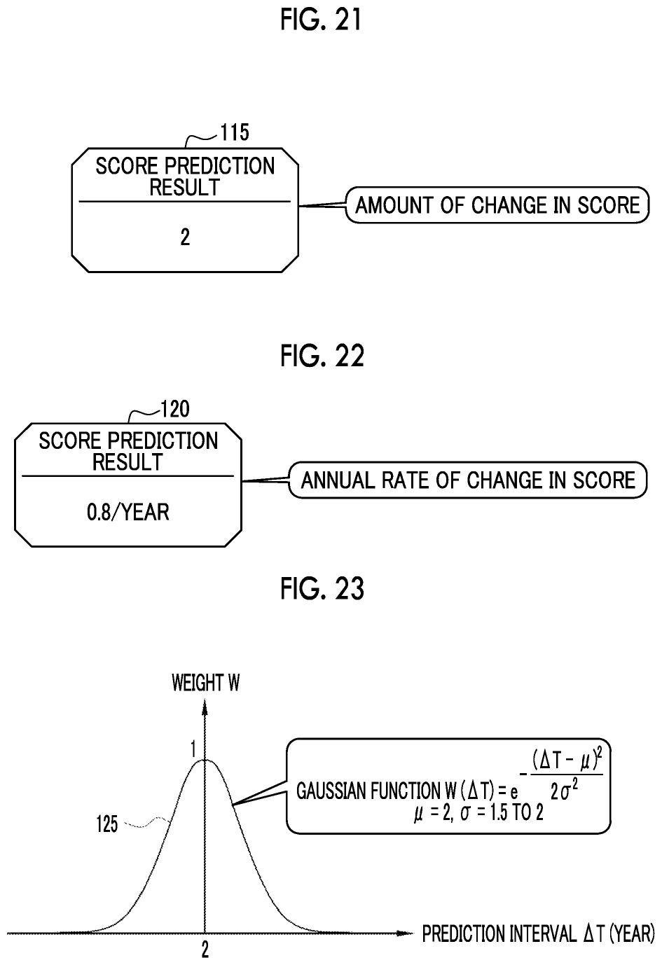

An example has been described in which the progression prediction result 18 or 105 or the integrated progression prediction result 110 is distributed to the user terminal 11 as the prediction result regarding the dementia of the subject, but the present disclosure is not limited thereto. As the prediction result regarding the dementia of the subject, the score prediction result may be distributed to the user terminal 11 instead of or in addition to the progression prediction result. The score prediction result may be the score prediction result 59 indicating the cognitive ability test score 25 itself according to the first embodiment, and may be a score prediction result 115 shown in FIG. 21 as an example, and a score prediction result 120 shown in FIG. 22 as an example.

The score prediction result 115 shown in FIG. 21 indicates the amount of change in the cognitive ability test score 25. By adding this amount of change to the cognitive ability test score 25 of the target input data 16 input to the dementia progression prediction model 41 or subtracting this amount of change from the cognitive ability test score 25, the cognitive ability test score 25 at the future point in time can be calculated. In FIG. 21, 2 is exemplified as the amount of change. Therefore, by adding 2 to the cognitive ability test score 25 of the target input data 16 input to the dementia progression prediction model 41, the cognitive ability test score 25 at the future point in time is calculated.

The score prediction result 120 shown in FIG. 22 indicates an annual rate of change in the cognitive ability test score 25. The annual rate of change is a rate that indicates how much the cognitive ability test score 25 changes in one year. By multiplying this amount of change by the prediction interval 17 and adding the multiplication result to the cognitive ability test score 25 of the target input data 16 input to the dementia progression prediction model 41 or subtracting the multiplication result from the cognitive ability test score 25, the cognitive ability test score 25 at the future point in time can be calculated. In FIG. 22, 0.8/year is exemplified as the annual rate of change. Therefore, by multiplying 0.8 by the prediction interval 17 and adding the multiplication result to the cognitive ability test score 25 of the target input data 16 input to the dementia progression prediction model 41, the cognitive ability test score 25 at the future point in time is calculated. In a case where the prediction interval 17 is, for example, two years, the multiplication result is 0.8×2=1.6. In addition, in a case where the prediction interval 17 is, for example, two years and six months, the multiplication result is 0.8×2.5=2.

For the score prediction result 115 and the score prediction result 120 as well, the reference point in time of the prediction interval 17 may be the current point in time or the past point in time. Further, the plurality of score prediction results 115 or the plurality of score prediction results 120 are output from the dementia progression prediction model 41, and an integrated score prediction result obtained by integrating the plurality of score prediction results 115 or the plurality of score prediction results 120 may be derived. In this case, the integrated score prediction result may be derived by the arithmetic mean, or as exemplified in a fifth embodiment below, the integrated score prediction result may be derived by the weighted average.

### Fifth Embodiment

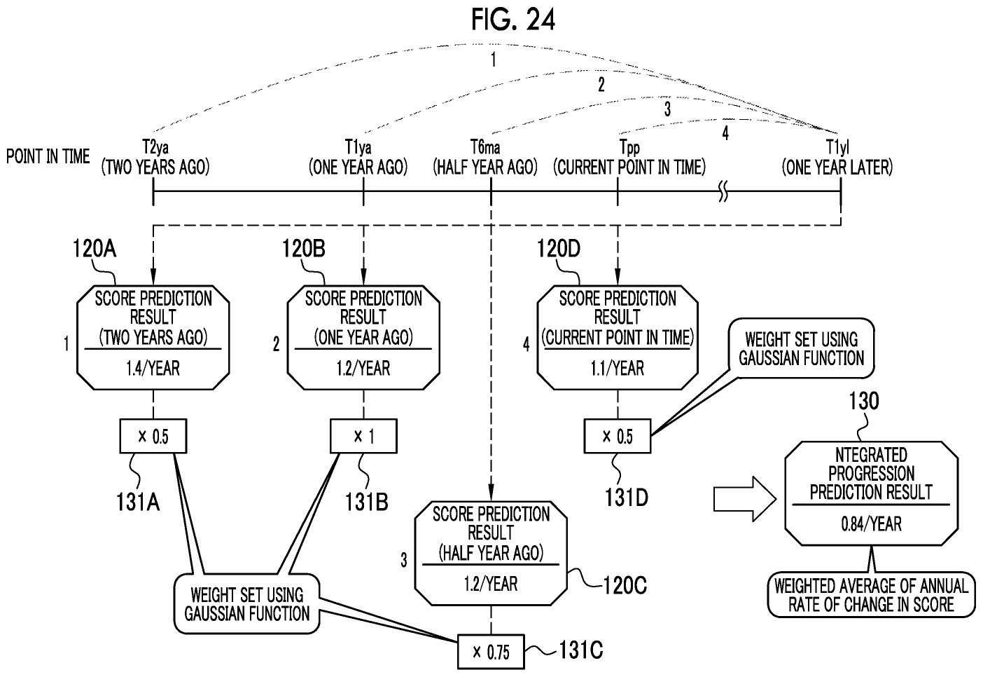

In the case of the score prediction result 120, a weighted average method shown in FIG. 23 or FIG. 24 can be employed as an example of a method of deriving an integrated score prediction result 130 in a case where a plurality of score prediction results 120 are output from the dementia progression prediction model 41. The integrated score prediction result 130 is an example of an “integrated prediction result” according to the technology of the present disclosure.

In FIG. 23, the weights given to the plurality of score prediction results 120 in a case where the weighted average is calculated are set using a Gaussian function 125. The Gaussian function 125 is an exponential function that has the prediction interval 17 (ΔT (year)) as a variable and has a weight W as a solution, as shown in a balloon of FIG. 23 and Equation (2) below. The Gaussian function 125 is an example of a “function having a prediction interval as a variable” according to the technology of the present disclosure. Note that μ is an average and α is a standard deviation, where μ=2, and σ=1.5 to 2. The reason why μ=2 is set is that it is empirically known that the reliability of the score prediction result 120 (annual rate of change of the cognitive ability test score 25) in a case where the prediction interval 17 is two years is relatively high.

$$W ( Δ T ) = {e}^{- \frac{{( Δ T - μ )}^{2}}{2 {σ}^{2}}} \qquad ( 2 )$$

In FIG. 24, a score prediction result 120A is output from the dementia progression prediction model 41 with respect to the input of the target input data 16 two years ago (denoted as T2ya in FIG. 24) and the prediction interval 17 from two years ago to one year later (three years), which are not shown. A score prediction result 120B is output from the dementia progression prediction model 41 with respect to the input of the target input data 16 one year ago and the prediction interval 17 from one year ago to one year later (two years), which are not shown. A score prediction result 120C is output from the dementia progression prediction model 41 with respect to the input of the target input data 16 half a year ago and the prediction interval 17 from half a year ago to one year later (one year and six months), which are not shown. A score prediction result 120D is output from the dementia progression prediction model 41 with respect to the input of the target input data 16 at the current point in time and the prediction interval 17 from the current point in time to one year later (one year), which are not shown.

In FIG. 24, the prediction unit 47 sets the weighted average of the annual rates of change of the score prediction results 120A to 120D as the integrated score prediction result 130. In the score prediction result 120A, since the prediction interval 17 is three years from two years ago to one year later, 0.5 is set as a weight 131A in a case where the weighted average is calculated, and in the score prediction result 120B, since the prediction interval 17 is two years from one year ago to one year later, 1, which is the highest, is set as a weight 131B. In the score prediction result 120C, since the prediction interval 17 is one year and six months from half a year ago to one year later, 0.75 is set as a weight 131C, and in the score prediction result 120D, since the prediction interval 17 is one year from the current point in time to one year later, 0.5 is set as a weight 131D. These weights 131A to 131D are set using the Gaussian function 125.

In FIG. 24, since the score prediction result 120A is 1.4/year, the score prediction result 120B is 1.2/year, the score prediction result 120C is 1.2/year, and the score prediction result 120D is 1.1/year, the integrated score prediction result 130 is (1.4×0.5+1.2×1+1.2×0.75+1.1×0.5)/4≈0.84/year.

In a case where a weight 131 is set using the Gaussian function 125 shown in FIG. 23, the score prediction results 120 (the score prediction result 120A two years ago and the score prediction result 120D at the current point in time) with relatively low prediction accuracy are less likely to be reflected in the integrated score prediction result 130. In other words, the score prediction results 120 (the score prediction result 120B one year ago and the score prediction result 120C half a year ago) with relatively high prediction accuracy are likely to be reflected in the integrated score prediction result 130. Therefore, a more reliable integrated score prediction result 130 can be derived. Other functions such as a triangular function may be used instead of the Gaussian function 125.

Instead of distributing the progression prediction result 18 and the like from the dementia progression prediction server 10 to the user terminal 11, screen data and the like of the dementia progression prediction screen 80 shown in FIG. 13 may be distributed from the dementia progression prediction server 10 to the user terminal 11.

The aspect of providing the progression prediction result 18 and the like for viewing by the doctor is not limited to the dementia progression prediction screen 80. A printed matter such as the progression prediction result 18 and the like may be provided to the doctor, or an e-mail to which the progression prediction result 18 and the like are attached may be transmitted to a mobile terminal of the doctor.

The progression prediction result is not limited to Alzheimer's dementia, and more generally, the progression prediction result may be a content that a subject is any one of normal control, preclinical AD, mild cognitive impairment, or dementia. Subjective cognitive impairment (SCI) and/or subjective cognitive decline (SCD) may be added as a prediction target. In addition, the progression prediction result may include a content that the subject develops Alzheimer's dementia two years later or does not develop Alzheimer's dementia two years later. In addition, for example, the progression prediction result may include a content that a degree of progression of the subject to dementia three years later is fast or slow. Further, the progression prediction result may include a content indicating whether the subject progresses to MCI from normal control or preclinical AD or whether the subject progresses to Alzheimer's dementia from normal control, preclinical AD, or MCI.

The learning of the dementia progression prediction model 41 shown in FIG. 8 may be performed in the dementia progression prediction server 10, or may be performed by a device other than the dementia progression prediction server 10. In addition, the learning of the dementia progression prediction model 41 may be continued even after the operation. In a case where the dementia progression prediction server 10 trains the dementia progression prediction model 41, the dementia progression prediction server 10 is an example of a “learning device” according to the technology of the present disclosure. In a case where a device other than the dementia progression prediction server 10 trains the dementia progression prediction model 41, the device other than the dementia progression prediction server 10 is an example of a “learning device” according to the technology of the present disclosure.

The dementia progression prediction server 10 may be installed in each medical facility or may be installed in a data center independent of the medical facility. In addition, the user terminal 11 may take some or all functions of each of the processing units 45 to 48 of the dementia progression prediction server 10.

The cognitive ability test score 25 may be a rivermead behavioural memory test (RBMT) score, an activities of daily living (ADL) score, or the like. Also, the cognitive ability test score 25 may be an ADAS-Cog score, a mini-mental state examination score, or the like.

The CSF test result 26 is not limited to the amount of p-tau 181 described as an example. The CSF test result 26 may be the amount of t-tau (total tau protein) or the amount of Aβ42 (amyloid β protein).

The MRI image 28 may be an image obtained by cutting out a portion of the brain, such as an image of a portion of a hippocampus. Also, a PET image or a SPECT image may be used as the test data 21 instead of or in addition to the MRI image 28.

As disclosed in WO2022/071158A, the progression prediction result 18 may be output from the dementia progression prediction model 41 by, for example, extracting an image of an anatomical region of a brain, such as a hippocampus, from a medical image such as the MRI image 28, inputting the extracted image of the anatomical region to a feature amount derivation model such as a convolutional neural network to output the feature amount through a convolution operation or the like, and inputting the feature amount to the dementia progression prediction model 41 as the target input data 16. The feature amount well represents a shape of the anatomical region and a feature of a texture, such as a degree of atrophy of a hippocampus. Therefore, the prediction accuracy of the progression prediction result 18 can be further improved. The image of the anatomical region to be extracted is not limited to the image of the hippocampus, and preferably includes a plurality of images of other anatomical regions, such as a parahippocampal gyrus, a frontal lobe, an anterior temporal lobe (anterior part of a temporal lobe), an occipital lobe, a thalamus, a hypothalamus, and an amygdala. The image of the anatomical region to be extracted preferably includes at least an image of a hippocampus, and more preferably includes at least an image of a hippocampus and an image of an anterior temporal lobe. In this case, the feature amount derivation model is prepared for each of images of a plurality of anatomical regions. In this manner, the aspect of extracting an image of an anatomical region of a brain from a medical image, inputting the extracted image of the anatomical region to a feature amount derivation model to output the feature amount, and inputting the feature amount to the dementia progression prediction model 41 as the target input data 16 is particularly effective for predicting progression from MCI.

The prediction regarding dementia includes a prediction of a cognitive function, such as how much the cognitive function of the subject is reduced after, for example, two years, a prediction of a risk of developing dementia, such as a degree of the risk of developing dementia of the subject, and the like.

Although dementia has been exemplified as a disease, the present disclosure is not limited thereto. The disease may be, for example, cerebral infarction. The target input data 16 in this case includes a National Institutes of Health Stroke Scale (hereinafter abbreviated as NIHSS) score, a Japan Stroke Scale (hereinafter abbreviated as JSS) score, a CT image, an MRI image, and the like. In addition, the machine learning model is not limited to the machine learning model in which the plurality of types of target input data 16 related to the disease are input, such as the dementia progression prediction model 41. In this way, the medical support may be progression prediction and/or diagnosis support for diseases other than dementia. The disease may be cerebral infarction, which is exemplified, or neurodegenerative disease such as Parkinson's disease, and cranial nerve disease including cerebrovascular disease.

However, dementia has become a social problem with the advent of an aging society in recent years. For this reason, it can be said that the dementia progression prediction server 10 using the dementia progression prediction model 41 to which the target input data 16 related to dementia is input has a form that matches the current social problem.

In each of the above embodiments, for example, as hardware structures of processing units that execute various kinds of processing, such as the reception unit 45, the RW control unit 46, the prediction unit 47, and the distribution control unit 48, various processors shown below can be used. As described above, in addition to the CPU 32 which is a general-purpose processor that functions as various processing units by executing software (operation program 40), the various processors include a programmable logic device (PLD), which is a processor capable of changing a circuit configuration after manufacture, such as a field programmable gate array (FPGA), and a dedicated electrical circuit, which is a processor having a circuit configuration specifically designed to execute specific processing such as an application specific integrated circuit (ASIC).

One processing unit may be configured by one of the various processors, or may be configured by a combination of the same or different kinds of two or more processors (for example, a combination of a plurality of FPGAs and/or a combination of the CPU and the FPGA). In addition, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. Second, as represented by a system on chip (SoC) or the like, there is a form of using a processor for realizing the function of the entire system including a plurality of processing units with one integrated circuit (IC) chip. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

In the technology of the present disclosure, the above-described various embodiments and/or various modification examples may be combined with each other as appropriate. In addition, the present disclosure is not limited to each of the above-described embodiments, and various configurations can be used without departing from the gist of the present disclosure. Furthermore, the technology of the present disclosure extends to a storage medium that non-transitorily stores a program, in addition to the program.

The described contents and illustrated contents shown above are detailed descriptions of the parts related to the technology of the present disclosure, and are merely an example of the technology of the present disclosure. For example, the above description of the configuration, function, operation, and effect is an example of the configuration, function, operation, and effect of the parts according to the technology of the present disclosure. Therefore, needless to say, unnecessary parts may be deleted, new elements may be added, or replacements may be made to the described contents and illustrated contents shown above within a range that does not deviate from the gist of the technology of the present disclosure. Further, in order to avoid complications and facilitate understanding of the parts related to the technology of the present disclosure, descriptions of common general knowledge and the like that do not require special descriptions for enabling the implementation of the technology of the present disclosure are omitted, in the described contents and illustrated contents shown above.

In the present specification, the term “A and/or B” is synonymous with the term “at least one of A or B”. That is, the term “A and/or B” means only A, only B, or a combination of A and B. In addition, in the present specification, the same approach as “A and/or B” is applied to a case where three or more matters are represented by connecting the matters with “and/or”.

All documents, patent applications, and technical standards described in the present specification are incorporated in the present specification by reference to the same extent as in a case where each of the documents, patent applications, technical standards are specifically and individually indicated to be incorporated by reference.

What is claimed is:

1. A medical support device comprising:
a processor; and
a memory connected to or built into the processor,
wherein the processor is configured to:
acquire target input data which is input data related to a disease of a subject whose progression of the disease is to be predicted, and a prediction interval which is an interval from a reference point in time to a future point in time at which prediction is performed; and
input the target input data and the prediction interval to a machine learning model trained using supervised training data including accumulated input data related to a disease at two or more points in time and a time interval of the input data, and cause the machine learning model to output a prediction result regarding the disease of the subject at the future point in time.

2. The medical support device according to claim 1,
wherein the input data includes at least one of test data indicating a result of a test related to a disease or diagnostic data indicating a result of a diagnosis related to the disease.

3. The medical support device according to claim 1,
wherein the target input data includes data at a current point in time of the subject, and
the reference point in time includes the current point in time.

4. The medical support device according to claim 1,
wherein the target input data includes data at a past point in time of the subject, and
the reference point in time includes the past point in time.

5. The medical support device according to claim 1,
wherein the processor is configured to, in a case where a plurality of pieces of the target input data and a plurality of the prediction intervals corresponding to a plurality of the reference points in time are acquired,
cause the machine learning model to output a plurality of the prediction results for each of the plurality of pieces of target input data and the plurality of prediction intervals, and
derive an integrated prediction result in which the plurality of prediction results are integrated.

6. The medical support device according to claim 5,
wherein the processor is configured to derive an arithmetic mean of the plurality of prediction results as the integrated prediction result.

7. The medical support device according to claim 5,
wherein the processor is configured to derive a weighted average of the plurality of prediction results as the integrated prediction result.

8. The medical support device according to claim 7,
wherein the processor is configured to change weights given to the plurality of prediction results in a case where the weighted average is calculated, according to the prediction interval.

9. The medical support device according to claim 8,
wherein the processor is configured to set the weights given to the plurality of prediction results in the case where the weighted average is calculated, using a function having the prediction interval as a variable.

10. The medical support device according to claim 1,
wherein the disease is dementia.

11. An operation method of a medical support device, the method comprising:
acquiring target input data which is input data related to a disease of a subject whose progression of the disease is to be predicted, and a prediction interval which is an interval from a reference point in time to a future point in time at which prediction is performed; and
inputting the target input data and the prediction interval to a machine learning model trained using supervised training data including accumulated input data related to a disease at two or more points in time and a time interval of the input data, and causing the machine learning model to output a prediction result regarding the disease of the subject at the future point in time.

12. A non-transitory computer-readable storage medium storing an operation program of a medical support device causing a computer to execute a process comprising:
acquiring target input data which is input data related to a disease of a subject whose progression of the disease is to be predicted, and a prediction interval which is an interval from a reference point in time to a future point in time at which prediction is performed; and
inputting the target input data and the prediction interval to a machine learning model trained using supervised training data including accumulated input data related to a disease at two or more points in time and a time interval of the input data, and causing the machine learning model to output a prediction result regarding the disease of the subject at the future point in time.

13. A learning device that performs learning,
the learning device being configured to, using at least accumulated input data related to a disease at two or more points in time and a time interval of the input data, as supervised training data, and using target input data which is input data related to a disease of a subject whose progression of the disease is to be predicted, and a prediction interval which is an interval from a reference point in time to a future point in time at which prediction is performed, as inputs,
learn to obtain a prediction result regarding the disease of the subject at the future point in time, as an output.

14. A learning method comprising:
learning, using at least accumulated input data related to a disease at two or more points in time and a time interval of the input data, as supervised training data, and using target input data which is input data related to a disease of a subject whose progression of the disease is to be predicted, and a prediction interval which is an interval from a reference point in time to a future point in time at which prediction is performed, as inputs, to obtain a prediction result regarding the disease of the subject at the future point in time, as an output.
